(19) United States
(12) Patent Application Publication
(10) Pub. No.: US 2014/0066781 A1
(43) Pub. Date: Mar. 6, 2014

(54) MEDICAL DIAGNOSIS DEVICE AND METHOD FOR CONTROLLING THE DEVICE

(71) Applicant: Electronics and Telecommunications Research Institute, (US)

(72) Inventors: Hyoung-Jun PARK, Gwangju-si (KR); Hyun-Seo KANG, Gwangju-si (KR); Keo-Sik KIM, Gwangju-si (KR); Young-Sun KIM, Daejeon-si (KR)

(73) Assignee: Electronics and Telecommunications Research Institute, Daejeon-si (KR)

(21) Appl. No.: 13/870,546

(22) Filed: Apr. 25, 2013

(30) Foreign Application Priority Data
Aug. 28, 2012 (KR) 10-2012-0094426

(51) Int. Cl. A61B 1/00

(52) U.S. Cl. CPC A61B 1/00009
USPC 600/475; 600/473

(57) ABSTRACT

A medical diagnosis device and a method for controlling the device that detect an abnormal portion of a diagnosis target early and accurately are provided. A medical diagnosis device according to an embodiment of the present invention includes: a light source configured to irradiate light onto a diagnosis target; an optical filter configured to filter out visible light and infrared light from light reflected from the diagnosis target and convert an optical path of the filtered visible or infrared light; a polarization beam splitter configured to polarize the infrared light filtered by the optical filter; a first image acquisition unit configured to acquire a first image from the visible light filtered by the optical fiber; and a second image acquisition unit configured to acquire a second image from the infrared light polarized by the polarization beam splitter.

### CROSS-REFERENCE TO RELATED APPLICATION

This application claims the benefit under 35 U.S.C. §119(a) of a Korean Patent Application No. 10-2012-0094426, filed on Aug. 28, 2012, the entire disclosure of which is incorporated herein by reference for all purposes.

### BACKGROUND

1. Field

The following description relates to a medical diagnosis device and a method for controlling the device, and more particularly, to a medical image diagnosis technology.

2. Description of the Related Art

An endoscope, one of medical diagnosis devices, is usually used to look inside a body cavity or organ for the purpose of a medical examination. The endoscope is referred to as a bronchoscope, gastroscope, laparoscope, colonoscope, etc. depending on the body part it examines, and is inserted directly into an organ to observe the organ with its imaging device and perform a medical diagnosis and operation. However, the conventional endoscope provides only a surface image of a diagnosis target and thus has limitations in sensing a minute change in an organ, for example, thermal distribution, bloodstream change, etc. Furthermore, the endoscope also has limitations in that it may be difficult to quickly check a problem of the interior of the organ caused by a disease and to visually observe a lesion of an inspection target depending on the disease.

To overcome the above-described problems of the endoscope, various methods are being proposed. An endoscope having a structure of acquiring endoscope images by irradiating a light at the end of the probe and then collecting a light reflected by a tissue through the optical fiber guide in the endoscope camera module has been proposed. Also, an endoscope including a function of irradiating an ultraviolet ray onto a tissue of an inspection target using an optical fiber bundle and analyzing a reflected wavelength to determine the abnormality of the tissue has been proposed. Also, an endoscope configured to generate photoluminescence on a tissue having an absorbed photosensitizer using an excitation light and read the photoluminescence to determine the abnormality of the tissue has been proposed.

### SUMMARY

The following description relates to a medical diagnosis device and a method for controlling the device, which can detect an abnormal portion of a diagnosis target early and accurately.

In one general aspect, a medical diagnosis device according to an embodiment of the present invention includes: a light source configured to irradiate light onto a diagnosis target; an optical filter configured to filter out visible light and infrared light from light reflected from the diagnosis target and convert an optical path of the filtered visible or infrared light; a polarization beam splitter configured to polarize the infrared light filtered by the optical filter; a first image acquisition unit configured to acquire a first image from the visible light filtered by the optical fiber; and a second image acquisition unit configured to acquire a second image from the infrared light polarized by the polarization beam splitter.

In another general aspect, a method of controlling a medical diagnosis device includes: irradiating light onto a diagnosis target; filtering out visible light and infrared light from light reflected from the diagnosis target and converting an optical path of the filtered visible or infrared light; polarizing the filtered infrared light; and acquiring a first image from the filtered visible light and acquiring a second image from the polarized infrared light.

Other features and aspects will be apparent from the following detailed description, the drawings, and the claims.

### BRIEF DESCRIPTION OF THE DRAWINGS

FIG. 1 is a block diagram of a medical diagnosis device according to an embodiment of the present invention.

FIGS. 2A and 2B show an external appearance and an optical path of an optical filter according to an embodiment of the present invention, respectively.

FIGS. 3A and 3B show an external appearance and an optical path of an optical filter according to another embodiment of the present invention, respectively.

FIGS. 4A and 4B show a configuration for controlling an optical filter and an external appearance of the optical filter according to another embodiment of the present invention, respectively.

FIG. 5 is a reference view showing an optical characteristic of an optical filter according to an embodiment of the present invention.

FIG. 6 is a flowchart showing a method of controlling a medical diagnosis device according to an embodiment of the present invention.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Hereinafter, embodiments of the present invention will be described in detail with reference to the accompanying drawings. In the following description, when the detailed description of the relevant known function or configuration is determined to unnecessarily obscure the important point of the present invention, the detailed description will be omitted. Moreover, the terms that have been defined as described above may be altered according to the intent of a user or operator, or conventional practice. Therefore, the terms should be defined on the basis of the entire content of this specification.

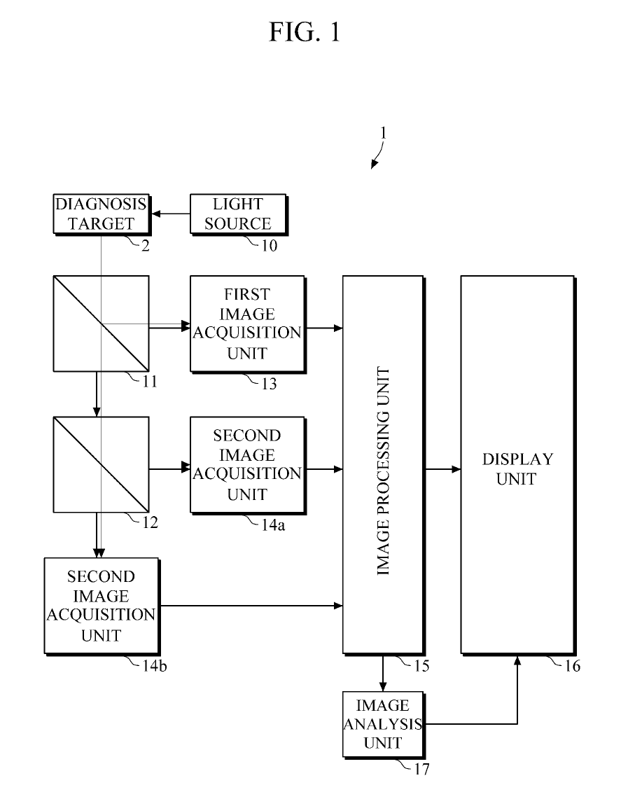

FIG. 1 is a block diagram of a medical diagnosis device 1 according to an embodiment of the present invention.

Referring to FIG. 1, the medical diagnosis device 1 includes a light source 10, an optical filter 11, a polarization beam splitter 12, a first image acquisition unit 13, and second image acquisition units 14a and 14b, and may further include an image processing unit 15, a display unit 16, and an image analysis unit 17.

The medical diagnosis device 1 is a device for diagnosis and treatment in the medical field, which is intended to monitor a human body, i.e., a diagnosis target, to diagnose or treat a disease. The diagnosis target may be a human organ. In this case, the medical diagnosis device 1 can be inserted into the body. The medical diagnosis device 1 may be an endoscope. For example, the endoscope is used to monitor a bronchus, a stomach, a colon, etc. to diagnose and/or treat a disease of the organ. For convenience of description, the endoscope will be described below as the medical diagnosis device 1 of the present invention. However, the present invention is not limited thereto.

The light source 10 irradiates light onto a diagnosis target 2. The diagnosis target 2 may be a human organ, such as a bronchus, a stomach, a colon, etc. The light source may be provided inside or at the head of the endoscope. The light source 10 may be a white light source, such as halogen lamp, xenon lamp, light emitting diode (LED), light bulb, etc.

The optical filter 11 filters out a visible light and an infrared light from a light reflected from the diagnosis target 2 and then converts an optical path of the visible light or infrared light. When the light source 10 irradiates light onto the diagnosis target 2, the diagnosis target 2 absorbs some light and reflects some light. At this point, the reflected light is input to the optical filter 11. Then, the optical filter 11 separates visible light and infrared light from the input light and then converts an optical path of the visible light or infrared light.

The optical filter according to an embodiment of the present invention, as shown in FIG. 1, reflects the visible light (400-750 nm) of the input light to convert the optical path in a direction perpendicular to a direction of the input light, and transmits the infrared light (800-1,650 nm) in an interface of the optical filter 11 without the optical path being converted. In this case, the optical filter 11 has an optical characteristic of reflecting the visible light and transmitting the infrared light with respect to a wavelength of 700 nm.

According to another embodiment of the present invention, The optical filter according to an embodiment of the present invention, as shown in FIG. 1, reflects the visible light (400-750 nm) of the input light to convert the optical path in a direction perpendicular to a direction of the input light, and transmits the infrared light (800-1,650 nm) in an interface of the optical filter 11 without the optical path being converted. In this case, the optical filter 11 has an optical characteristic of reflecting the visible light and transmitting the infrared light with respect to a wavelength of 700 nm.

The visible light separated by the optical filter 11 is input to a first image acquisition unit 13. The first image acquisition unit 13 acquires a first image using the visible light input from the optical filter 11. The first image may be a surface image of the human organ. Unlike this, the infrared light separated by the optical filter 11 is used for the second image acquisition units 14a and 14b to acquire a second image. The second image may be a thermal distribution image.

As described above, according to an embodiment of the present invention, the optical filter 11 separates the visible light and the infrared light from the input light, the first image acquisition unit 13 acquires the surface image of the human organ using the visible light, and the second image acquisition units 14a and 14b acquire the thermal distribution image using the infrared light. Thus, an abnormal portion of the diagnosis target may be detected early and accurately. It is difficult to determine a normal portion and an abnormal portion using only the external surface image. However, according to an embodiment of the present invention, the thermal distribution image may be acquired using the infrared light, thereby detecting an abnormal portion that has not been detected in the surface image acquired from the visible light. The abnormal portion may be a lesion or wound portion. The lesion is an area of abnormal change caused by disease.

The polarization beam splitter (hereinafter referred to as PBS) 12 receives infrared light from the optical filter to polarize the received infrared light. In this case, the PBS 12 may separate the infrared light received from the optical filter 11 into components polarized in a specific direction using a polarization characteristic and a phase relation between the components. For example, the PBS 12 separates the infrared light into p-polarized light and s-polarized light with respect to an input direction of the infrared light. Mostly, the s-polarized light is reflected, and the p-polarized light is transmitted. The p-polarized light corresponds to light polarized on a plane of incidence of the PBS 12. The s-polarized light corresponds to light polarized perpendicularly to a plane of incidence of the PBS 12. The plane of incidence is a plane limited by the reflected light ray and perpendicular to a reflection surface. The PBS 12 may be formed inclined at 45 degrees from one side of a cube.

The reason for using the PBS 12 is to accurately identify a normal portion and an abnormal portion of the diagnosis target and detect the abnormal portion early. That is, by acquiring a thermal distribution image with a polarization characteristic of the infrared light being applied thereto using the PBS 12, in addition to a thermal distribution image from the infrared light using the optical filter 11, the detection accuracy can be increased and the detection time can be shortened. When a variety of thermal distribution images are acquired depending on change in a polarization component of the infrared light, an abnormal portion that has not been detected in the thermal distribution image without the change in the polarization component may be detected.

The first image acquisition unit 13 receives the filtered visible light from the optical filter 11 and acquires a first image through photoelectric conversion of the received visible light. The first image may be a surface image of the human organ. The first image acquisition unit 13 may be a charge-coupled device (hereinafter referred to as CCD) camera. The first image acquisition unit 13 may have a visible light blocking filter removed therefrom.

The second image acquisition units 14a and 14b receive the polarized infrared light from the PBS 12 and acquire a second image through photoelectric conversion of the received infrared light. The second image acquisition units 14a and 14b may each have a CCD camera with an infrared light blocking filter removed therefrom. The second image may be a thermal distribution image of the organ, which may be acquired using both p-polarized infrared light and s-polarized infrared light. Image acquisition processes of the first image acquisition unit 13 and the second image acquisition units 14a and 14b may be performed simultaneously or separately.

The image processing unit 15 is configured as a standard signal processing circuit for processing the images acquired by the first image acquisition unit 13 and the second image acquisition units 14a and 14b. For example, the image processing unit 15 may include a pre-amplifier, a correlated double sampling (CDS) circuit, an analog-to-digital converter (ADC), and a digital signal processor (DSP).

According to an embodiment of the present invention, the image processing unit 15 combines the second images acquired by the second image acquisition units 14a and 14b. For example, when the infrared light is polarized by the PBS 12 into p-polarized light and s-polarized light, the image processing unit 15 combines a thermal distribution image acquired from the p-polarized light and a thermal distribution image acquired from the s-polarized light by the second image acquisition units 14a and 14b. According to another embodiment of the present invention, the image processing unit 15 combines a surface image of the organ generated by the first image acquisition unit 13 and thermal distribution images generated by the second image acquisition units 14a and 14b.

The image processing unit 15 transmits a result of image processing to the display unit 16 or the image analysis unit 17. In this case, the analysis unit 17 may determine an abnormal portion using comparison between images. When the result is displayed by the display unit 16, a diagnostician may determine an abnormal portion using comparison between images.

The image analysis unit 17 analyzes images acquired by the first image acquisition unit 13 and the second image acquisition units 14a and 14b to detect an abnormal portion of the diagnosis target 2. According to an embodiment of the present invention, the image analysis unit 17 compares the thermal distribution images acquired by the second image acquisition units 14a and 14b to determine a portion having a difference between the images as the abnormal portion. That is, the thermal distribution image acquired from the p-polarized infrared light may be different from the thermal distribution image acquired from the s-polarized infrared light depending on a polarization characteristic and a phase relation between components of the infrared light. In this case, the image analysis unit 17 may determine a portion with a difference as the abnormal portion.

The display unit 16 outputs the images acquired by the first image acquisition unit 13 and the second image acquisition units 14a and 14b or an image formed by combing the images. Furthermore, the display unit 16 may output a result of abnormal-portion determination from the image analysis unit 17.

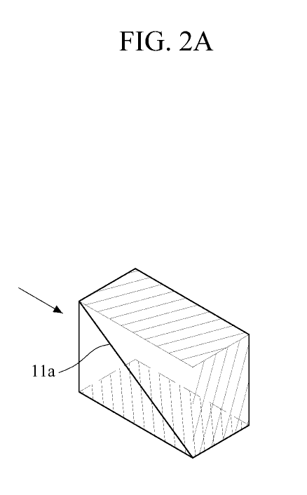

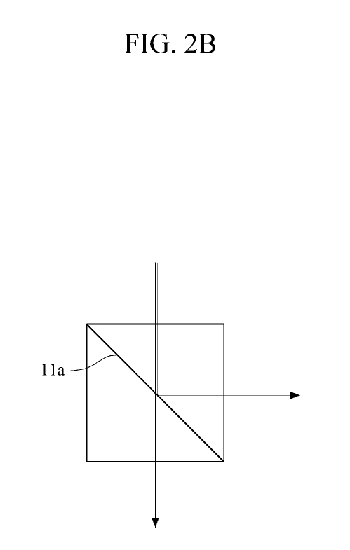

FIGS. 2A and 2B are reference views showing an external appearance and an optical path of an optical filter 11 according to an embodiment of the present invention, respectively.

Referring to FIGS. 2A and 2B, the optical filter 11 is formed as a cube to facilitate image acquisition of the first image acquisition unit 13 and the second image acquisition units 14a and 14b. In this case, a cube type wavelength division multiplexing (hereinafter referred to as WDM) filter 11a having light transmittance may be coated on a 45 degree inclined plane of one side of the cube. Also, in order to maintain a light transmittance characteristic and remove noise from images acquired by the first image acquisition unit 13 and the second image acquisition units 14a and 14b, an anti-reflection (AR) coating may be performed on other sides of the cube. Three oblique-striped sides of FIG. 2A are planes on which the AR coating is processed.

According to an embodiment of the present invention, as shown in FIG. 2B, the cube type WDM filter 11a reflects the visible light (400-750 nm) to convert the optical path in a direction perpendicular to a direction of the input light, and transmits the infrared light (800-1,650 nm) in an interface of the cube type WDM filter 11a without the optical path being converted. In this case, the cube type WDM filter 11a has an optical characteristic of reflecting the visible light and transmitting the infrared light with respect to a wavelength of 700 nm.

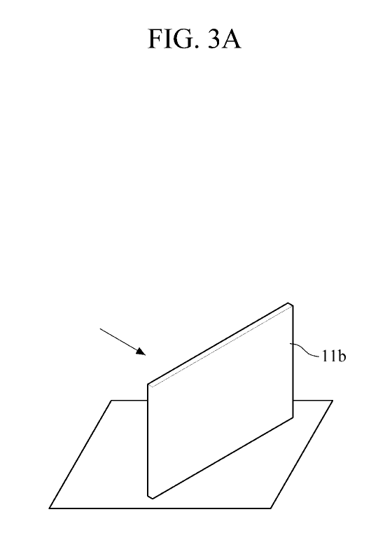

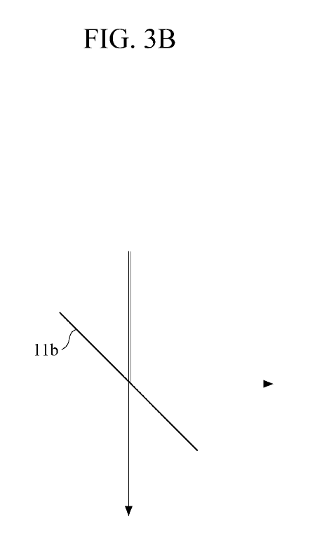

FIGS. 3A and 3B are reference views showing an external appearance and an optical path of an optical filter 11 according to another embodiment of the present invention, respectively.

Referring to FIGS. 3A and 3B, the optical filter may be a thin film type WDM filter 11b. In this case, in order to have the same transmittance and reflectance characteristics as the cube type WDM filter 11a, the thin film type WDM filter 11b is fixed to be inclined at 45 degrees. A filter holder for fixing the thin film type WDM filter 11b may be in a structure in which a rectangular groove is processed to be inclined at 45 degrees with respect to a plane, as shown in FIG. 3a. For example, in the processing of the thin film type WDM filter 11b, the thin film type WDM filter 11b is inserted into the rectangular groove of the filter holder, coated with ultraviolet (UV) epoxy, and then fixed using a UV curing device. The configuration of the optical filter 11 may be implemented relatively simply with the structure and process for the filter holder, and may be manufactured at a low cost as compared with the cube type WDM filter 11a of FIG. 2A.

According to an embodiment of the present invention, as shown in FIG. 3B, the thin film type WDM filter 11b reflects the visible light (400-750 nm) to convert the optical path in a direction perpendicular to a direction of the input light, and transmits the infrared light (800-1,650 nm) in an interface of the thin film type WDM filter 11b without the optical path being converted. In this case, the thin film type WDM filter 11b has an optical characteristic of reflecting the visible light and transmitting the infrared light with respect to a wavelength of 700 nm.

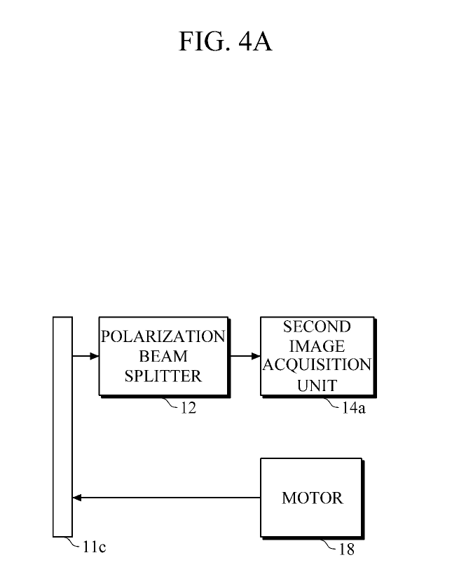

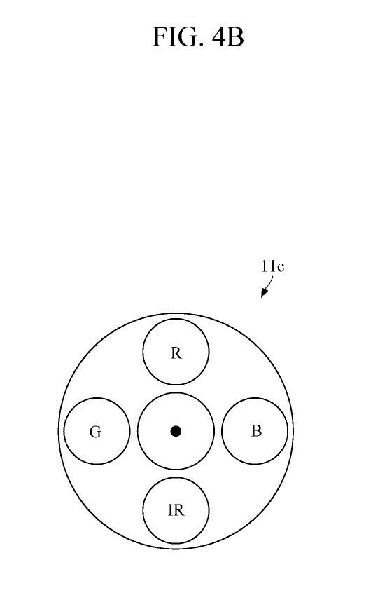

FIGS. 4A and 4B are reference views showing a configuration for controlling the optical filter 11 and an external appearance of the optical filter 11 according to another embodiment of the present invention, respectively.

Referring to FIGS. 4A and 4B, the optical filter 11 may be a rotation type optical filter 11c. The rotation type optical filter 11c includes a red light filter (R), a green light filter (G), a blue light filter (B), and an infrared light filter, and sequentially transmits light selected by rotation of a motor 18. The rotation type optical filter 11c has the filters with different light transmittances which are each fixed in a hole. The rotation type optical filter 11c is rotated by the motor 18.

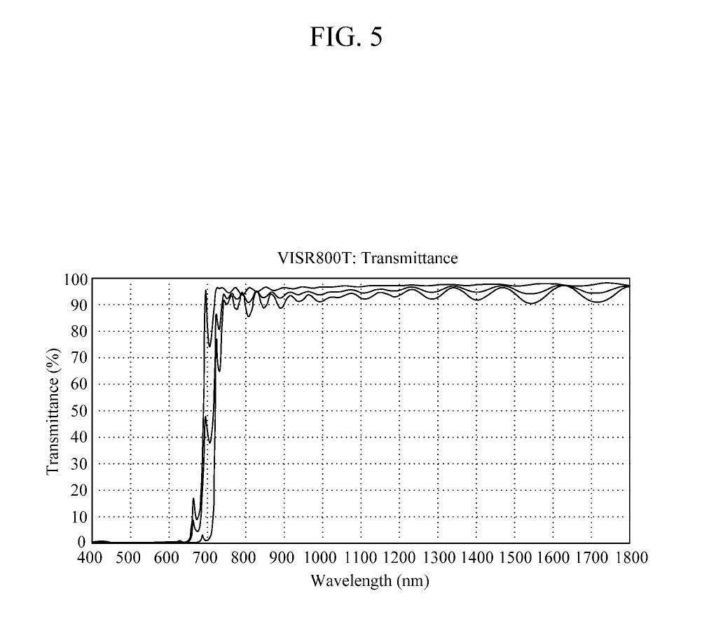

FIG. 5 is a reference view showing an optical characteristic of the optical filter 11 according to an embodiment of the present invention.

Referring to FIG. 5, the optical filter 11 has transmittance and reflectance characteristics. For example, the optical filter 11 has an optical characteristic of reflecting the visible light and transmitting the infrared light with respect to a wavelength of 700 nm.

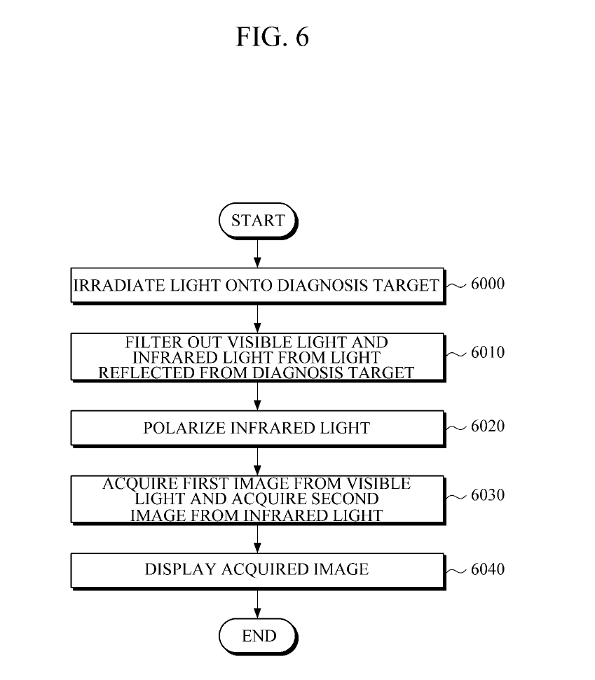

FIG. 6 is a flowchart showing a method of controlling the medical diagnosis device 1 according to an embodiment.

Referring to FIGS. 1 and 6, the medical diagnosis device 1 irradiates light onto a diagnosis target through the light source 10 (6000). Next, the optical filter 11 filters out visible light and infrared light from light reflected from the diagnosis target 2 and then converts an optical path of the filtered visible light or infrared light (6010).

Next, the PBS 12 polarizes the infrared light filtered by the optical filter 11 (6020). In operation 6020, the PBS 12 may separate the filtered infrared light into components polarized in a specific direction using a polarization characteristic and a phase relation between the components. For example, the PBS 12 may separate the filtered infrared light into p-polarized light and s-polarized light with respect to the input direction of the filtered infrared light.

Next, the first image acquisition unit 13 acquires a first image from visible light filtered by the optical filter 11, and the second image acquisition units 14a and 14b acquire second images from infrared light polarized by the PBS 12 (6030).

According to an additional embodiment of the present invention, the image analysis unit 17 analyzes the first image acquired by the first image acquisition unit 13 and the second images acquired by the second image acquisition units 14a and 14b to detect an abnormal portion of the diagnosis target 2. In this operation, the image analysis unit 17 compares the second images with each other to determine a portion having a difference between the images as the abnormal portion. According to an additional embodiment of the present invention, the display unit 16 outputs the first and second images or an image formed by combining the first and second images (6040).

According to an embodiment of the present invention, the surface image and thermal is distribution image of the diagnosis target can be acquired using transmittance and reflectance characteristics of light wavelength, and thereby the abnormal portion of the inspection target can be detected early and accurately using the acquired images. That is, the thermal distribution image can be acquired using the infrared light separated from light reflected from the inspection target. Thus, an abnormal portion that has not been detected in the surface image acquired using the visible light can be detected early in the thermal distribution image.

Furthermore, the polarization characteristic of the infrared light may be applied to the thermal distribution image, thereby further enhancing accuracy and efficiency in detection of the abnormal portion. That is, with a variety of thermal distribution images depending on change in a polarization component of the infrared light, an abnormal portion that has not been detected in the thermal distribution image without the change in the polarization component can be detected early.

While this invention has been particularly shown and described with reference to preferred embodiments thereof, it will be understood by those skilled in the art that various changes in form and details may be made therein without departing from the spirit and scope of the invention as defined by the appended claims. Therefore, the preferred embodiments should be considered in descriptive sense only and not for purposes of limitation. Therefore, the scope of the invention is defined not by the detailed description of the invention but by the appended claims, and all differences within the scope will be construed as being included in the present invention.

What is claimed is:

1. A medical diagnosis device comprising:
a light source configured to irradiate light onto a diagnosis target;
an optical filter configured to filter out visible light and infrared light from light reflected from the diagnosis target and convert an optical path of the filtered visible or infrared light;
a polarization beam splitter configured to polarize the infrared light filtered by the optical filter;
a first image acquisition unit configured to acquire a first image from the visible light filtered by the optical fiber; and
second image acquisition units each configured to acquire a second image from the infrared light polarized by the polarization beam splitter.

2. The medical diagnosis device of claim 1, wherein the optical filter is formed as a cube to facilitate image acquisition of the first image acquisition unit and the second image acquisition units.

3. The medical diagnosis device of claim 2, wherein the optical filter has one side of the cube that is coated with a wavelength division multiplexing filter having a light transmittance and other sides of the cube that are anti-reflection coated to maintain a light transmittance characteristic and remove a noise component from images acquired by the first image acquisition unit and the second image acquisition units.

4. The medical diagnosis device of claim 1, wherein the optical filter is a wavelength division multiplexing thin film filter.

5. The medical diagnosis device of claim 1, wherein the optical filter is a rotation type optical filter configured to have a red light filter, a green light filter, a blue light filter, and an infrared light filter, and sequentially transmits light selected by rotation of a motor.

6. The medical diagnosis device of claim 1, wherein the polarization beam splitter receives the infrared light from the optical filter and separates the received infrared light into components polarized in a specific direction using a polarization characteristic and a phase relation between the components.

7. The medical diagnosis device of claim 6, wherein the polarization beam splitter separates the received infrared light into p-polarized light and s-polarized light with respect to an input direction.

8. The medical diagnosis device of claim 1, wherein the polarization beam splitter is formed as a cube.

9. The medical diagnosis device of claim 1, wherein the first image acquisition unit and the second image acquisition units are each a charge-coupled device camera.

10. The medical diagnosis device of claim 1, further comprising: an image processing unit configured to combine the second images acquired by the second image acquisition units and combine the combined second images with the first image generated by the first image acquisition unit.

11. The medical diagnosis device of claim 1, further comprising an image analysis unit configured to detect an abnormal portion of the diagnosis target using the images acquired by the first image acquisition unit and the second image acquisition units.

12. The medical diagnosis device of claim 11, wherein the image analysis unit is configured to compare the second images acquired by the second image acquisition units to determine a portion having a difference between the second images as an abnormal portion.

13. The medical diagnosis device of claim 1, further comprising a display unit configured to output the images acquired by the first image acquisition unit and the second image acquisition units or an image formed by combining the images.

14. The medical diagnosis device of claim 1, wherein the medical diagnosis device is an endoscope.

15. A method of controlling a medical diagnosis device, the method comprising:
irradiating light onto a diagnosis target;
filtering out visible light and infrared light from light reflected from the diagnosis target and converting an optical path of the filtered visible or infrared light;
polarizing the filtered infrared light; and
acquiring a first image from the filtered visible light and acquiring a second image from the polarized infrared light.

16. The method of claim 15, wherein the polarizing comprises separating the filtered infrared light into components polarized in a specific direction using a polarization characteristic and a phase relation between the components.

17. The method of claim 16, wherein the polarizing comprises separating the filtered infrared light into p-polarized light and s-polarized light with respect to an input direction of the filtered infrared light.

18. The method of claim 15, further comprising detecting an abnormal portion of the diagnosis target using the acquired first and second images.

19. The method of claim 18, wherein the detecting comprises comparing the acquired second images to determine a portion having a difference between the second images as an abnormal portion.

20. The method of claim 15, further comprising outputting the acquired first and second images or an image formed by combining the acquired first and second images.
